(19) United States
(12) Patent Application Publication
(10) Pub. No.: US 2013/0225640 A1
(43) Pub. Date: Aug. 29, 2013

(54) CYANOACRYLATE-BASED BIO-ADHESIVE COMPOSITION

(71) Applicant: Dong Jin Kim, Andong-si (KR)

(72) Inventor: Dong Jin Kim, Andong-si (KR)

(73) Assignee: INTUITIVE MEDI CORP INC., Daegu-metro (KR)

(21) Appl. No.: 13/883,162

(22) PCT Filed: Nov. 2, 2011
(86) PCT No.: PCT/KR2011/008283

§ 371 Date: May 2, 2013

(30) Foreign Application Priority Data
Nov. 2, 2010 (KR) 10-2010-0108334

(51) Int. Cl. A61K 31/4422

(52) U.S. Cl. CPC A61K 31/4422
USPC 514/345

(57) ABSTRACT

The present invention relates to a cyanoacrylate-based bio-adhesive composition, and aims to provide a cyanoacrylate-based bio-adhesive composition having an excellent biodegradability and anti-bacterial effect. The present invention provides a cyanoacrylate-based bio-adhesive composition having an enhanced biodegradability by using, as a thickening agent, poly octyl cyanoacrylate generated as a byproduct when preparing octyl cyanoacrylate, and having an excellent anti-bacterial effect by using 3,5-diiodo-4-pyridone-1-acetic acid as an anion stabilizer.

### TECHNICAL FIELD

The present invention relates to a cyanoacrylate-based bio-adhesive composition and, more particularly, to a cyanoacrylate-based bio-adhesive composition having an enhanced biodegradability by using, as a thickening agent, poly octyl cyanoacrylate produced as a byproduct at the preparation of octyl cyanoacrylate, and having an excellent anti-bacterial effect by using 3,5-diiodo-4-pyridone-1-acetic acid as an anion stabilizer.

### BACKGROUND ART

A tissue adhesive is a high-tech medical product which is useful to join damaged tissues by means of simple manipulations in place of a suture. Normally tissues to be joined are in a wet state by body fluid or blood, so it is difficult for a tissue adhesive to obtain sufficient adhesive strength. Also, a tissue adhesive should have a rapid adhesive property and biocompatibility. For these reasons, just limited materials have been used for a tissue adhesive. Fibrin glue and cyanoacrylate adhesive are more practical than other materials. Fibrin glue is composed of fibrinogen, thrombin and calcium chloride, and a reaction among them creates an adhesive strength. However, fibrinogen causes a possibility of a virus infection since it is separated from blood plasma of the human body. Although fibrin glue has been widely used for many parts due to its hemostasis property and good tissue regeneration, it has relatively poor adhesive strength in comparison with cyanoacrylate adhesive.

Cyanoacrylate adhesive is cyanoacrylate ester monomer and originally known as instant glue. Cyanoacrylate adhesive which is a colorless liquid is instantaneously polymerized by means of anionic polymerization under weak base such as water or amine. Compared to methyl cyanoacrylate having short molecular chain, cyanoacrylate of long alkyl ester has better adhesive strength in blood and also is slowly decomposed. While methyl cyanoacrylate is rapidly hydrolyzed and causes toxic infection by creating formaldehyde, octyl cyanoacrylate (brand name: Dermabond) which has recently received FDA approval has good histocompatibility and hence is expected to be increasingly applied to a tissue adhesive. However, octyl cyanoacrylate has a lower production yield and is mostly selling at a high price. Therefore, the commercial use of octyl cyanoacrylate is restricted considerably.

### DETAILED DESCRIPTION OF THE INVENTION

### Technical Problems

Accordingly, the present inventor tried to manufacture a cost-effective cyanoacrylate-based adhesive having an excellent biodegradability and anti-bacterial effect. As a result, the present inventor found that it is possible to manufacture a cost-effective cyanoacrylate-based bio-adhesive composition having an enhanced biodegradability by using, as a thickening agent, poly octyl cyanoacrylate produced as a byproduct at the preparation of octyl cyanoacrylate and having an excellent anti-bacterial effect by using 3,5-diiodo-4-pyridone-1-acetic acid as an anion stabilizer, and then completed this invention.

Thus, an object of the present invention is to provide a cost-effective cyanoacrylate-based bio-adhesive component that has an excellent biodegradability and anti-bacterial effect.

### Technical Solutions

In order to accomplish the above-mentioned object, the present invention provides a cyanoacrylate-based bio-adhesive composition that includes poly octyl cyanoacrylate as a thickening agent, and 3,5-diiodo-4-pyridone-1-acetic acid as an anion stabilizer.

In the cyanoacrylate-based bio-adhesive composition, the poly octyl cyanoacrylate may be manufactured through steps of preparing octyl cyanoacetate by reacting cyanoacetic acid and octane alcohol at an equivalence ratio of 1:1 under the existence of a solvent, reacting the octyl cyanoacetate and paraformaldehyde at an equivalence ratio of 1:1 under the existence of a solvent, isolating and refining octyl cyanoacrylate after removing the solvent when the reaction is completed, and obtaining poly octyl cyanoacrylate from a byproduct left by refinement of the octyl cyanoacrylate.

Additionally, the cyanoacrylate-based bio-adhesive composition may be manufactured as wound dressing prosthesis, cartilage adhesive, organ adhesive, blood vessel adhesive, nerve adhesive, or oral adhesive.

### Advantageous Effects

The bio-adhesive composition of this invention has an enhanced biodegradability by using, as a thickening agent, poly octyl cyanoacrylate which is left as a byproduct after octyl cyanoacrylate is isolated and refined, and has an excellent anti-bacterial effect by using 3,5-diiodo-4-pyridone-1-acetic acid as an anion stabilizer.

### DESCRIPTION OF THE DRAWING

FIG. 1 is a flow diagram showing a method for manufacturing octyl cyanoacrylate and poly octyl cyanoacrylate used in the present invention.

### MODE FOR CARRYING OUT THE INVENTION

Hereinafter, embodiments of the present invention will be described in detail. The following descriptions are provided to assist in a comprehensive understanding of the embodiments. Well known techniques will be omitted to avoid obscuring the subject matter of the present invention.

The meaning of specific terms or words used in the specification and the claims should not be limited to the literal or commonly employed sense, but should be construed in accordance with the spirit of the invention as described herein. The description of the various embodiments is to be construed as exemplary only and does not describe every possible instance of the invention. Therefore, it should be understood that various changes may be made and equivalents may be substituted for various elements of the invention.

The present invention relates to a cost-effective cyanoacrylate-based bio-adhesive composition which has an enhanced biodegradability by using, as a thickening agent, poly octyl cyanoacrylate produced as a byproduct at the preparation of octyl cyanoacrylate, and which has an excellent anti-bacterial effect by using 3,5-diiodo-4-pyridone-1-acetic acid as an anion stabilizer.

Poly octyl cyanoacrylate used as a thickening agent in this invention is a byproduct which is produced at the preparation of octy cyanoacrylate and typically discarded.

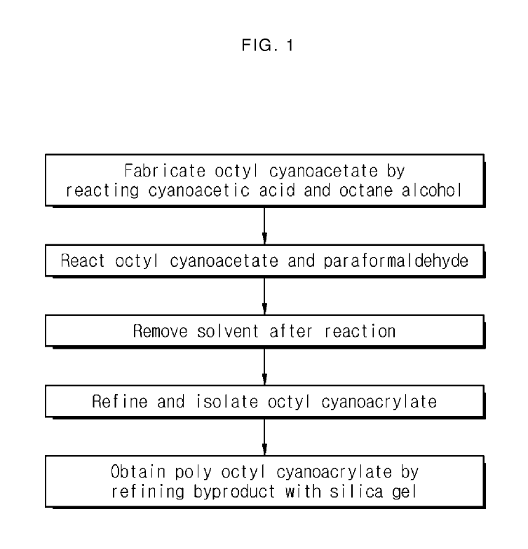

A method for manufacturing poly octyl cyanoacrylate used in this invention is shown in FIG. 1. Specifically, the manufacturing method includes a step of preparing octyl cyanoacetate by reacting cyanoacetic acid and octane alcohol at an equivalence ratio of 1:1 under the existence of a solvent; a step of reacting the octyl cyanoacetate and paraformaldehyde at an equivalence ratio of 1:1 under the existence of a solvent; a step of isolating and refining octyl cyanoacrylate after removing the solvent when the reaction is completed; and a step of obtaining poly octyl cyanoacrylate from a byproduct left by refinement of the octyl cyanoacrylate. A solvent used in each reaction process is organic solvent typically used in the art, including benzene, toluene, xylan, or the like, but not limited thereto. Additionally, poly octyl cyanoacrylate may be obtained by refining the byproduct with silica gel, and preferably the content thereof is 0.001˜4 weight % with regard to the total weight of the composition.

In this invention, 3,5-diiodo-4-pyridone-1-acetic acid used as an anion stabilizer has a structure of chemical formula 1 below and has an anti-bacterial effect as well as an anion stabilizing function. Preferably, the anion stabilizer is added at 0.8˜2.9 weight % with regard to the total weight of the composition.

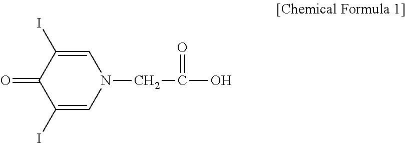 [SMILES: O=C(O)Cn1cc(I)c(=O)c(I)c1]

Additionally, the cyanoacrylate-based bio-adhesive composition of this invention contains at least one polymerizable cyanoacrylate monomer as a main ingredient, and in addition to the thickening agent and the anion stabilizer, may further contain any other ingredients typically used at the manufacture of bio-adhesive in the art. For example, in addition to at least one polymerizable cyanoacrylate monomer, the thickening agent and the anion stabilizer, the cyanoacrylate-based bio-adhesive composition of this invention may further include at least one of a free radical stabilizer, an anionic vapor phase stabilizer, a polymerization initiator, a plasticizer, a colorant, a preservative, a heat dispersant, a thixotropes, and a biocompatible agent. Specifically, with regard to the total weight of the composition, at least one polymerizable cyanoacrylate monomer of 90˜95 weight %, a free radical stabilizer of 0.01˜0.2 weight %, a polymerization initiator of 0.01˜2 weight %, and other additives (a plasticizer, a colorant, a preservative, a heat dispersant, or a biocompatible agent) of 0.001˜2 weight % may be contained.

In an embodiment of this invention, the at least one polymerizable cyanoacrylate monomer may use one or more selected from the group consisting of octyl cyanoacrylate, dodecyl cyanoacrylate, 2-ethylhexyl cyanoacrylate, butyl cyanoacrylate, ethyl cyanoacrylate, methyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, butyl lactoyl cyanoacrylate, butyl glycoloyl cyanoacrylate, isopropyl glycoloyl cyanoacrylate, ethyl lactoyl cyanoacrylate, ethyl glycoloyl cyanoacrylate, isopropyoxy ethyl cyanoacrylate, methoxy butyl cyanoacrylate, and mixtures thereof. Preferably, the at least one polymerizable cyanoacrylate monomer uses octyl cyanoacrylate.

The free radical stabilizer may use hydroquinone, hydroquinone monomethyl ether, methoxy hydroquinone, catechol, pyrogallol, benzoquinone, 2-hydroxybenzoquinone, p-methoxy phenol, t-butyl catechol, butylated hydroxyl anisole, butylated hydroxyl toluene, t-butyl hydroquinone, and mixture or blend thereof.

The anionic vapor phase stabilizer may be sulphur dioxide, boron trifluoride, or hydrogen fluoride.

The polymerization initiator may use quaternary ammonium salts, for example, domiphen bromide, butyrylcholine chloride, benzalkonium bromide, benzalkonium chloride, acetyl choline chloride, or the like.

The plasticizer may include acetyl tributyl citrate, dimethyl sebacate, dibutyl sebacate, triethyl phosphate, tri(2-ehtylhexyl)phosphate, tri(p-cresyl)phosphate, glyceryl triacetate, glyceryl tributyrate, diethyl sebacate,dioctyl adipate, isopropyl myristate, butyl stearate, lauric acid, trioctyl trimellitate, dioctyl glutarate, poly dimethyl siloxane, and mixture thereof.

The preservative may be methylparaben, methylparaben sodium, ethylparaben, propylparaben, proplyparaben sodium, butylparaben, cresol, chlorocresol, or the like.

The heat dispersant may use, alone or in combinations, potassium nitrate, sodium acetate trihydrate, sodium sulfate decahydrate, barium hydroxide octahydrate, calcium oxalate dehydrate, magnesium oxalate dehydrate, aluminum hydroxide, zinc sulfate, aluminum oxide, barium oxide, titanium oxide, manganese oxide, calcium oxide, copper, lead, nickel, aluminum, zinc, carbon black, carbides, urea, paraffin wax, polyvinyl fluoride, 2-hydroxy-2-trimethylsylanyl-propionitrile, 1-fluorophensacyclo[6.3.0.02,6.03,10.05,9]undecane, 6,7-diazabicyclo[3.2.1]-6-octene, 5,5,6,6-tetramethylbicyclo[2.2.1]heptane-2-ol, complex of dimethyl magnesium and trimethyl aluminum, N-benzyl-2,2,3,3,4,4,4-heptafluoro-butyramide, 3-isopropyl-5,8a-dimethyl-decahydronaphthalene-2-ol, 2-hydroxymethyl-1,7,7-trimethyl-bicyclo[2.2.1]heptane-2-ol, 3,5-dichloro-3-methyl-cyclophentane-1,2-dione, (5-methyl-2-oxo-bicyclo[3.3.1]non-3-en-1-yl)-acetic acid, 4b,6a,11,12-tetrahydro-indeno[2,1-a]fluorene-5,5,6,6-tetracarbonitrile, tetraccsafluoro-tetradecahydro-anthracene, 4,5-dichlorobenzene-1,2-dicarbaldehyde, bicyclo[4.3.1]dec-3-en-8-one, 3-tert-butyl-1,2-bis-(3,5-dimethylphenyl)-3-hydroxyguanidine, 1-[2,6-dihydroxy-4-methoxy-3-methylphenyl]butan-1-one, 2,3,6,7-tetrachloronaphthalene, 2,3,6-trimethylnaphthalene, dodecafluoro-cyclohexane, 2,2,6,6-tetramethyl-4-hepten-3-one, 1,1,1-trichloro-2,2,2-trifluoro-ethane, [5-(9H-beta-carbolin-1-yl)-furan-2-yl]methanol, 5-nitro-benzo[1,2,3]thiadiazole, 4,5-dichloro-thiophene-2-carboxylic acid, 2,6-dimethyl-isonicotinonitrile, nonafluoro-2,6-bis-trifluoromethyl-piperidine, (dimethylamino)difluoroborane, dinitrogen pentoxide, chromyl fluoride, chromium hexacarbonyl, 1-methylcyclohexanol, phenyl ether, nonadecane, 1-tetradecanol, 4-ethylphenol, benzophenone, maleic anhydride, octacosane, dimethyl isophthalate, butylated hydroxytoluene, glycolic acid, vanillin, magnesium nitrate hexahydrate, cyclohexanone oxime, glutaric acid, D-sorbitol, phenanthrene, methacrylamide, fluorine, 4-hydroxybenzaldehyde, trans-stilbene, neopentyl glycol, pyrogallol, or diglycolic acid.

The biocompatible agent is ingredient to reduce the concentration levels of active formaldehyde produced during in-vivo biodegradation of the polymer, and may be sulfites, bisulfites, mixtures of sulfites and bisulfites, or the like.

The bio-adhesive composition of this invention may be used for, but not limited to, wound dressing prosthesis, cartilage adhesive, organ adhesive, blood vessel adhesive, nerve adhesive, oral adhesive, and the like.

Hereinafter, the present invention will be more fully described through embodiments. These embodiments are provided only to assist in understanding of the present invention, and the scope of this invention is not to be limited by such embodiments. Various well-known modifications, substitutions and additions may be made and they are included in the scope of the present invention.

### Embodiment 1] Manufacture of Octyl Cyanoacetate and Poly Octyl Cyanoacetate

A 3.5 L mixture in which cyanoacetic acid and octane alcohol were mixed at an equivalence ratio of 1:1 was put in a glass flask and then 3.5 L toluene was added as a solvent to react. After a reaction was completed, toluene was removed and octyl cyanoacetate was isolated and refined. At this time, the reaction yield was 80% or more.

A 3.5 L mixture in which the above octyl cyanoacetate and paraformaldehyde were mixed at an equivalence ratio of 1:1 was put in a glass flask and then 3.5 L toluene and base catalyst were added to react. After a reaction was completed, toluene was removed and octyl cyanoacrylate was isolated and refined. From a byproduct left after the octyl cyanoacrylate was refined, poly octyl cyanoacrylate was obtained and refined using a silica gel column. At this time, the yield of poly octyl cyanoacrylate was 80%.

### [Embodiment 2] Manufacture of Bio-Adhesive Composition

By mixing the octyl cyanoacrylate of 95 weight % and the poly octyl cyanoacrylate of 1.5 weight % obtained in the above embodiment 1 together with 3,5-diiodo-4-pyridone-1-acetic acid of 0.8 weight %, methoxy hydroquinone of 0.3 weight %, sulphur dioxide of 1.2 weight % and benzalkonium chloride of 1.2 weight %, a bio-adhesive composition was manufactured.

### EXPERIMENTAL EXAMPLE 1

### Property Test of Bio-Adhesive Composition

Properties of the bio-adhesive composition manufactured in the above embodiment 2 were measured by means of the following methods. Table 1 below shows the results.

<Adhesive Strength>

According to ASTM D1002 method using a tensile strength tester (Instron Inc.-4204), about 0.03 mL of the bio-adhesive in the embodiment 2 was adhered to a stainless steel with adhesion area 1 cm² and then an adhesive strength was measured after 24 hours had elapsed at a temperature of 37° C. and a relative humidity of 75% [adhesive strength=adhesive strength between metals (stainless steel)].

<Flexibility>

According to ASTM D3111method using a hardness tester D-type (Teclock Inc.-GS 709N), the bio-adhesive in the embodiment 2 was poured into a molder and cured for 72 hours at a temperature of 37° C. and a relative humidity of 75%. Then, the flexibility of a resultant sample with 2cm width and 5mm thickness was measured under a load of 5 kgf.

<Viscosity>

According to ASTM D2556 method using a viscometer (Brookfield Inc.-viscometer DV-II), a viscosity was measured at sp 15, 100 rpm under 23□0.5° C.

<Adhesion Time>

According to JIS K6861, 0.03 mL of the bio-adhesive was adhered to a stainless steel plate with adhesion area 1 cm³ by a load of 2 kgf and then the minimum durable time was measured under 5 kg weight.

TABLE 1

| | Adhesive | | Adhesion | |
|---|---|---|---|---|
| | Strength | Viscosity | Time | |
| Sample | (kgf/cm²) | (cP) | (sec) | Hardness |
| Embodiment | 145 | 55 | <25 | 61 |
| 2 | | | | |

As shown in Table 1, it was confirmed that the bio-adhesive of embodiment 2 had excellent properties including adhesive strength.

### EXPERIMENTAL EXAMPLE 2

### Antibiotic Test

In order to check the antimicrobial efficacy of the bio-adhesive composition manufactured in the embodiment 2, the anti-bacterial effect was tested against Staphylococcus aureus, ATCC 6538, Staphylococcus epidermidis, ATCC 51625, Enterococcus faecium, ATCC 700221, Escherichia coli, ATCC 8739, Pseudomonas aeruginosa, ATCC 9027, and Candida albicans, ATCC 10231.

The above microbes were grown in 20 ml sterile Tryptic Soy Broth for 16˜24 hours at 35˜37° C.

Trypticase Soy Agar (TSA) plates were used for the assay. A 0.85% saline solution was utilized for dilutions. All media was stream sterilized prior to use. Agar plates were prepared by pouring approximately 20 ml of molten media into sterile disposable petri dishes (100×15 mm). The agar plates were allowed to solidify under a laminar flow hood.

Overnight cultures of respective microbes were vortexed and a 1:100 dilution was prepared to obtain a minimum of 10⁴ colony-forming units (CFU)/ml. The diluted inoculum was spread on the agar plate surface using sterile cotton swabs. Care was taken to cover the entire agar surface uniformly. Plates were allowed to air dry for 30 minutes.

20 μl of the bio-adhesive composition in the embodiment 2 was added to the center of the inoculated plate. Two drops of control sample were expressed onto separate TSA plates. Test sample drops were not spread manually and were allowed to polymerize on the plates to form a thin film. The plates were incubated at 35˜37° C. for 24 hours. Plates were examined for inhibition zones [minimum inhibitory concentration (MIC) and minimum bactericidal concentration (MBC)] resulting from the diffusion of the active agents out of the product and into the agar medium. The inhibition zone was measured from the edge of the film to the edge where the clear zone ends. Table 2 below shows the results.

TABLE 2

DIMPTS (ppm)

| Cultured microbes | MIC | MBC |
|---|---|---|
| S. aureus (ATCC 6538) | 50 | <150 |
| S. epidermidis (ATCC 51625) | 10 | <100 |
| E. faecium (ATCC 700221) | 25 | NA |
| E. coli (ATCC 8739) | 90 | <150 |
| P. aeruginosa (ATCC 9027) | 100 | NA |
| C. albicans (ATCC 10231) | 25 | <100 |

As shown in Table 2, it was confirmed that the bio-adhesive of embodiment 2 had an anti-bacterial effect.

While this invention has been particularly shown and described with reference to embodiments thereof, it will be understood by those skilled in the art that various changes in form and details may be made therein without departing from the spirit and scope of the invention as defined by the appended claims.

What is claimed is:

1. A cyanoacrylate-based bio-adhesive composition comprising:
poly octyl cyanoacrylate as a thickening agent; and
3,5-diiodo-4-pyridone-1-acetic acid as an anion stabilizer.

2. The cyanoacrylate-based bio-adhesive composition of claim 1, wherein the poly octyl cyanoacrylate is manufactured through steps of:
preparing octyl cyanoacetate by reacting cyanoacetic acid and octane alcohol at an equivalence ratio of 1:1 under the existence of a solvent;
reacting the octyl cyanoacetate and paraformaldehyde at an equivalence ratio of 1:1 under the existence of a solvent;
isolating and refining octyl cyanoacrylate after removing the solvent when the reaction is completed; and
obtaining poly octyl cyanoacrylate from a byproduct left by refinement of the octyl cyanoacrylate.

3. The cyanoacrylate-based bio-adhesive composition of claim 1 is manufactured as wound dressing prosthesis, cartilage adhesive, organ adhesive, blood vessel adhesive, nerve adhesive, or oral adhesive.
